(19) United States
(12) Patent Application Publication
(10) Pub. No.: US 2010/0113784 A1
(43) Pub. Date: May 6, 2010

(54) PROCESS FOR PREPARING CRYSTALLINE ARIPIPRAZOLE

(76) Inventors: Niraj Shyamlal Shah, Gujarat (IN); Shriprakash Dhar Dwivedi, Gujarat (IN); Kishorkumar Maneklal Vinchhi, Gujarat (IN); Sathya Varahala Raju Nadimpally, Gujarat (IN)

Correspondence Address: LADAS & PARRY LLP, 26 WEST 61ST STREET, NEW YORK, NY, 10023, US

(21) Appl. No.: 12/443,299

(22) PCT Filed: Sep. 24, 2007
(86) PCT No.: PCT/IN2007/000440

§ 371 Date: Jan. 7, 2010

(30) Foreign Application Priority Data
Sep. 28, 2006 (IN) 1572/MUM/2006

(51) Int. Cl. C07D 401/12

(52) U.S. Cl. 544/363

(57) ABSTRACT

The present invention relates the process for preparation of stable polymorph of aripiprazole and in particular the present invention is related to an improved process for the preparation of crystalline anhydrous aripiprazole and its hydrochloride salt.

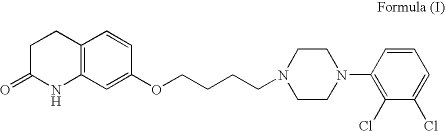

### FIELD OF THE INVENTION

The present invention relates the process for preparation of stable polymorph of aripiprazole and in particular the present invention is related to an improved process for the preparation of crystalline anhydrous aripiprazole.

### BACKGROUND OF THE INVENTION

Any discussion of the prior art throughout the specification should in no way be considered as an admission that such prior art is widely known or forms part of common general knowledge in the field.

The present invention relates to an improved process for the preparation of 7-[4-[4-(2,3-dichlorophenyl)-1-piperazinyl]butoxy]-2,3-dihydro-2-(1H)-quinolinone generally known as Aripiprazole. Aripiprazole has structural formula (I).

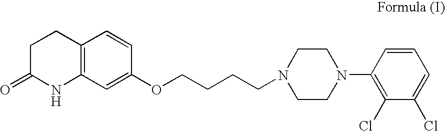

Aripiprazole is an atypical antipsychotic agent useful for the treatment of schizophrenia. Schizophrenia is a common type of psychosis characterized by delusions, hallucinations and extensive withdrawl from others. Onset of schizophrenia typically occurs between the age of 16 and 25 and affects 1 in 100 individuals worldwide. It is more prevalent than Alzheimer's disease, multiple sclerosis, insulin-dependent diabetes and muscular dystrophy. Early diagnosis and treatment can lead to significantly improved recovery and outcome. Moreover, early therapeutic intervention can avert costly hospitalization.

U.S. Pat. No. 4,734,416 and U.S. Pat. No. 5,006,528 discloses aripiprazole and processes for the preparation thereof. The said patents also disclose various salts of aripiprazole and their preparation. According to example 1 of Japanese patent publication No. 02-191256, anhydrous aripiprazole crystals are manufactured for example reacting 7-(4-bromo butoxy)-3,4-dihydrocarbostyril with 1-(2,3-dichlorophenyl)piperazine and recrystallizing the resulting raw anhydrous aripiprazole with ethanol. Also, according to the proceedings of the 4^{th} Japanese-Korean Symopsium on Separation Technology (Oct. 6-8, 1996), anhydrous aripiprazole crystals are manufactured by heating aripiprazole hydrate at 80° C. However, the anhydrous aripiprazole crystals obtained by the aforementioned methods have the disadvantages of being significantly hygroscopic.

Aripiprazole has been approved by the FDA for the treatment of schizophrenia in 2, 5, 10, 15, 20 and 30 mg tablets for oral administration and is currently marketed under the brand name of Abilify®. Notably, in the Summary Basis of Approval (SBA) of New Drug Application 21-436, the innovator stated that “[t]he current tablet formulation of aripiprazole makes use of anhydrous Form I drug substance.” Aripiprazole was also approved in the United Kingdom for treating schizophrenia. The European Public Assessment Report for Ability of the European Medicine Agency (EMEA) mentions the existence of polymorphs. In particular, the EMEA states that “[a]ripiprazole can exist in several crystalline forms, Form I was chosen for the development and commercialization.” According to the EMEA information, “[t]he formulation contains stable milled crystalline aripiprazole because of the limited solubility in water and the hydrophobic nature of the active substance.”

Until now, aripiprazole was known to exist in at least seven different crystalline forms.

As discussed in WO 2003/026659, which is incorporated herein by reference in its entirety, seven known polymorphs of aripiprazole are (1) hydrate Form A, (2) anhydrous Form B, (3) anhydrous Form C, (4) anhydrous Form D, (5) anhydrous Form E, (6) anhydrous Form F and (7) anhydrous Form G. The various forms differ from each other in their physical and spectroscopic properties as well as in their methods of preparation.

According to WO 2003/026659, these various forms of aripiprazole can be prepared in the following ways: Aripiprazole hydrate Form A has been obtained by milling conventional aripiprazole hydrate to a mean particle size of 50 μm or less.

Aripiprazole Form B has been prepared by several different processes including, for example: (a) drying aripiprazole hydrate Form A for 24 hours at 100° C. using a hot dryer, (b) drying aripiprazole hydrate Form A for 18 hours at 100° C. in a hot dryer and then heating for 3 hours at 120° C., (c) heating conventional hygroscopic aripiprazole anhydrous crystals or conventional aripiprazole hydrate at 100° C. or 120° C. for 3 to 50 hours.

Anhydrous Form C can be obtained by heating conventional anhydrous aripiprazole crystals at a temperature of about 145° C. to yield colorless prism crystals.

Anhydrous Form D is obtained by recrystallizing conventional anhydrous aripiprazole crystals from toluene to form colorless plate crystals.

Anhydrous Form E can be prepared by heating and dissolving conventional anhydrous aripiprazole crystals in acetonitrile and cooling the resulting product to form colorless needle crystals.

Anhydrous Form F can be obtained by heating a suspension of conventional anhydrous aripiprazole crystals in acetone to form colorless prism crystals.

Anhydrous Form G is obtained by maintaining a glassy state anhydrous aripiprazole in a sealed vessel at room temperature for approximately 6 months. The initial glassy state anhydrous aripiprazole can be obtained by heating and melting anhydrous aripiprazole crystals at 170° C.

WO 2004/083183 describes the preparation of two forms of aripiprazole, designated therein as Forms I and II. Form I is obtained by crystallization from acetone, ethyl acetate, methanol or ethanol. Form II is obtained by dissolving aripiprazole in tetrahydrofuran followed by vacuum drying at 25° C. or spray drying. A comparison of the data (e.g., the X-ray diffractogram) from WO 2004/083183 to the data in WO 2003/026659 suggests that Form I corresponds to anhydrous Form D and that Form II corresponds to the aripiprazole hydrate Form A. WO 2004/106322 also describes the preparation of several forms of aripiprazole, designated therein as Forms II, III and IV. Form II is obtained by contacting or crystallizing the product from isopropyl alcohol, isopropyl acetate, methanol or mixtures thereof. Form III is obtained by contacting or crystallizing the product from isobutyl acetate or ethanol, and Form IV is obtained by contacting or crystallizing the product from acetone, t-butyl alcohol and/or mixtures thereof or heating aripiprazole to about 150° C. A comparison of the data (e.g., the X-ray diffractograms) from WO 2004/106322 to the data in WO 2003/026659 suggests that Form II corresponds to the anhydrous Form D and that Form IV corresponds to Form C.

WO 2005/009990 describes the preparation of a crystalline non-hygroscopic form of aripiprazole designated therein as Form III and two novel solvates designated as aripiprazole methanolate Form IV and aripiprazole ethylene dichloride Form V. Form III is obtained by crystallization from a mixture of methyl t-butyl ether, acetonitrile and tetrahydrofuran. Aripiprazole methanolate Form IV is obtained by crystallization from a mixture of methanol and tetrahydrofuran. Aripiprazole ethylene dichloride Form V is obtained by crystallization from ethylene dichloride. Comparison of the X-ray diffractogram of Form HI from WO 2005/009990 to the above-described forms indicates it corresponds to the anhydrous Form D of WO 2003/026659.

During the Proceedings of the 4th Japan-Korean Symposium on Separation Technology (Oct. 6-8, 1996), it was disclosed that aripiprazole has two types of anhydrous polymorphs (type 1 and type 2) and a hydrous crystal (type 3). The anhydrous type 1 crystals of aripiprazole could be prepared by recrystallizing aripiprazole from an ethanol solution or by heating hydrous crystal type 3 at 80° C. Anhydrous crystal type 1 has a melting point of 140° C. The anhydrous type 2 crystals of aripiprazole could be prepared by heating anhydrous type 1 crystals of aripiprazole to 130 to 140° C. for 15 hours. The melting point of anhydrous type 2 crystals was 150° C. When anhydrous type 1 and 2 crystals of aripiprazole were recrystallized from an alcoholic solvent containing up to 20% (v/v) water, the crystals were converted to hydrous crystals type 3. WO 2005/058835 also describes anhydrous aripiprazole type 2, designated therein as Form II and herein as Form J, and methods of preparing it. According to U.S. Pat. No. 5,006,528, and as demonstrated in Scheme 1 (below), aripiprazole can be prepared by condensing 7-(4-bromobutoxy)-3,4-dihydro-2(1H)-quinolinone (i.e., Compound II; 7-(4-bromobutoxy)-3,4-dihydrocarbostyril) with 1-(2,3-dichlorophenyl)piperazine (i.e., Compound III) in acetonitrile under basic conditions (e.g., triethylamine) and in the presence of sodium iodide at reflux temperature. After removal of the solvent by evaporation, the resulting residue can be dissolved in chloroform, washed with water and dried with anhydrous magnesium sulphate. After removal of the solvent by evaporation, the residue can be recrystallized (twice) from ethanol to yield colorless flake crystals having a melting point of 139-139.5° C.

WO 03/026659 teaches the preparation of aripiprazole hydrate by dissolving the aripiprazole crude crystals in a hydrous organic solvent, subsequently heating followed by cooling the resulting solution. The organic solvent should be one, which is miscible with water, such as for example an alcohol, acetone, ether or a mixture thereof, with ethanol being particularly desirable. The amount of water in the hydrous solvent can be 10-25% by volume of the solvent, or preferably close to 20% by volume.

US2006012299 A1 claims the hemiethanolate and methanolate of aripiprazole. Hemiethanolate is converted to Form-B i.e. anhydrous aripiprazole on heating.

WO2007004061 A1 discloses the process for the preparation of Form J in ketonic solvents and Form L via Form H (hemiethanolate with 5% residual ethanol). Further drying of Form H converts to anhydrous aripiprazole.

Polymorphism is the occurrence of different crystalline forms of a single compound and it is a property of some compounds and complexes. Thus, polymorphs are distinct solids sharing the same molecular formula, yet each polymorph may have distinct physical properties. Therefore, a single compound may give rise to a variety of polymorphic forms where each form has different and distinct physical properties, such as different solubility profiles, different melting point temperatures and/or different x-ray diffraction peaks. Since the solubility of each polymorph may vary, identifying the existence of pharmaceutical polymorphs is essential for providing pharmaceuticals with predicable solubility profiles. It is desirable to investigate all solid state forms of a drug, including all polymorphic forms, and to determine the stability, dissolution and flow properties of each polymorphic form. Polymorphic forms of a compound can be distinguished in a laboratory by X-ray diffraction spectroscopy and by other methods such as, infrared spectrometry. For a general review of polymorphs and the pharmaceutical applications of polymorphs see G. M. Wall, Pharm Manuf. 3, 33 (1986); J. K. Haleblian and W. McCrone, J. Pharm. Sci., 58, 911 (1969); and J. K. Haleblian, J. Pharm. Sci., 64, 1269 (1975), all of which are incorporated herein by reference.

Accordingly, a feature of the present invention is giving a simple and easy handling process for the preparation of crystalline anhydrous aripiprazole with consistent results, meeting the criteria for hygroscopicity and stable.

Accordingly, a feature of the present invention is to provide a new crystalline Form of aripiprazole hydrochloride characterized by x-ray powder diffraction pattern and differential scanning calorimetry.

### OBJECTS OF THE INVENTION

It is an object of the present invention to overcome or ameliorate at least one of the disadvantages of the prior art, or to provide a useful alternative.

It is another object of the present invention in its preferred form to provide a simple and easy handling process for preparation of stable crystalline anhydrous aripiprazole.

Thus, it is an object of the present invention to provide a process for the preparation of crystalline anhydrous aripiprazole and crystalline aripiprazole hydrochloride Form-X.

### SUMMARY OF THE INVENTION

The present invention provides a process for preparing crystalline anhydrous aripiprazole comprising:
i. dissolving aripiprazole in an organic solvent;
ii. heating the solution from step (i) to temperatures of about 70° C. to about 90° C. to obtain clear solution;
iii. cooling the solution of step (ii) to about 25° C. to about 35° C.;
iv. cooling the solution of step (iii) to about 5° C. to about 10° C. and maintaining the temperatures as crystals form;
v. separating crystals from the solution;
vi. dissolving the wet crystals in an organic solvent;
vii. heating the solution from step (vi) to temperatures of about 70° C. to about 90° C. to obtain clear solution, optionally, characoalizing;
viii. seeding the solution of clear solution of step (vii) with crystalline anhydrous aripiprazole at temperatures of about 70° C.;
ix. cooling the solution of step (viii) to about 25° C. to about 35° C.;
x. cooling the solution of step (ix) to about 5° C. to about 10° C. and maintaining the temperatures as crystals form;
xi. separating the crystals from the solution; and
xii. drying the crystals at about 100° C. until the water content is not more than 0.5% w/w.

The present invention also relates to the process for the preparation of crystalline aripiprazole hydrochloride designated as “Form X”, which is characterized by x-ray powder diffraction pattern having 2θ values 3.84, 8.59, 11.62, 13.37, 15.35, 18.14, 22.02, 22.36, 23.11, 27.71 & 30.16 as the characteristic peaks and having DSC endotherm at 223° C.

### BRIEF DESCRIPTION OF THE DRAWINGS

FIG. 1 is a Differential Scanning Calorimetry (DSC) theromgram of crystalline anhydrous aripiprazole.

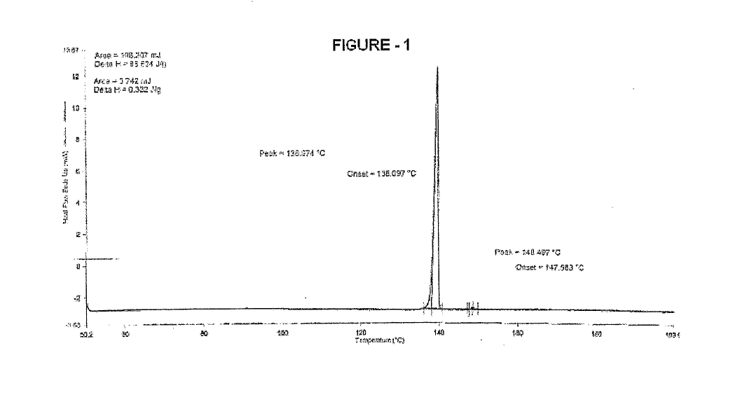

FIG. 2 is an X-ray powder diffractogram (XRD) of the crystalline anhydrous aripiprazole, measured on Rigaku D/Max-2200/PC Diffractometer with Cu K alpha-1 radiation source.

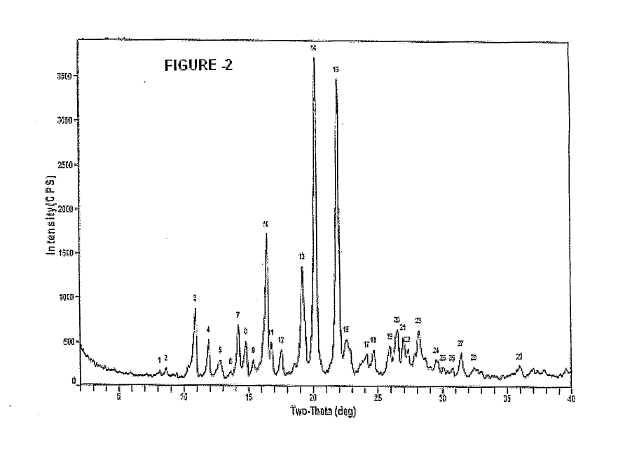

FIG. 3 is an X-ray powder diffractogram (XRD) of the aripiprazole hydrochloride “Form X”, measured on Rigaku D/Max-2200/PC Diffractometer with Cu K alpha-1 radiation source.

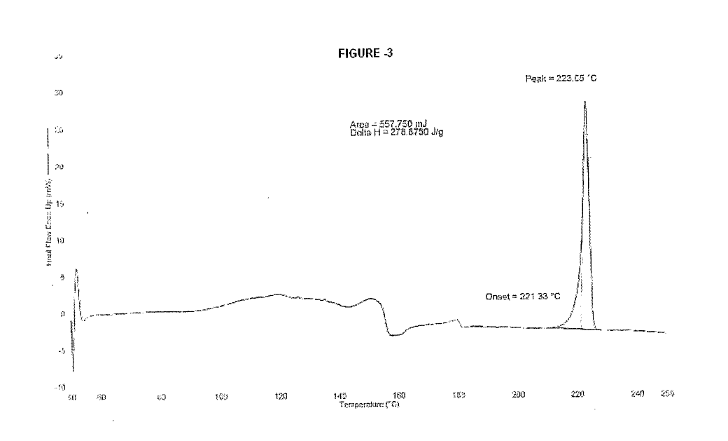

FIG. 4 is a Differential Scanning Calorimetry (DSC) theromgram of the present aripiprazole hydrochloride “Form X”.

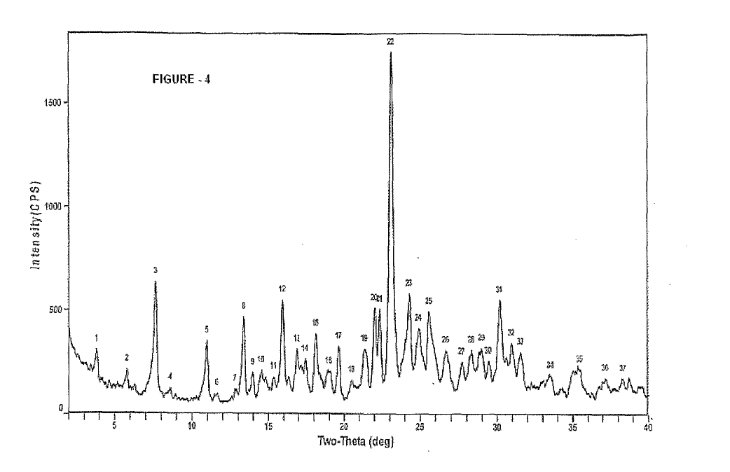

### DESCRIPTION OF THE INVENTION

As used here in the term “crystalline anhydrous aripiprazole” means it has substantially similar X-ray powder diffractogram as shown in FIG. II

The inventors of the present invention have discovered that the seeding results in obtaining the desired crystalline anhydrous aripiprazole in a consistent manner. The present invention provides an improved process for the preparation of crystalline anhydrous aripiprazole.

The crystalline anhydrous aripiprazole obtained by the process of the present invention can be identified by its DSC thermogram and XRPD analysis.

According to the first embodiment of the present invention, a process for preparing crystalline anhydrous aripiprazole comprising:
(i) dissolving aripiprazole in an organic solvent;
(ii) heating the solution from step (i) at about 70° C. to about 90° C. to obtain clear solution;
(iii) cooling the solution of step (ii) to about 25° C. to about 35° C., further to about 5° C. to about 10° C. and maintaining the temperatures as crystals form;
(iv) separating crystals from the solution;
(v) dissolving the wet crystals in an organic solvent;
(vi) heating the solution from step (v) at about 70° C. to about 90° C. to obtain clear solution;
(vii) optionally, charging 5%-10% decolorizing carbon to the clear solution of step (iv)
   and stirring at the heating temperature for 15-60 minutes;
(viii) removal of the carbon under the hot condition;
(ix) heating the filtrate of step (viii) under stirring to about 70° C. to about 90° C. to obtain clear solution;
(x) seeding the solution of step (ix) with crystalline anhydrous Aripiprazole at temperatures of about 70° C.;
(xi) cooling the solution of step (x) to about 25° C. to about 35° C.; further to about 5° C. to about 10° C. and maintaining the temperatures as crystals form;
(xii) filtering the separated solid in step (xi) further to about 0° C. to about 10° C. and washing the solid with organic solvent; and
(xiii) drying the solid of step (xii) at about 100° C. until the water content is not more than 0.5% w/w.

Preferably, the organic solvents that can be used for the preparation of crystalline anhydrous aripiprazole are alcohols such as methanol, ethanol, isopropanol, n-butanol and pentanol; or acetic acid, or tetrahydrofuran, or mixtures of any two or more thereof. The organic solvent preferably is ethanol.

Preferably, the aripiprazole dissolved in an organic solvent is heated at about 78° C. to about 85° C. to obtain clear solution.

Seeding the solution of step (vii) comprises adding crystalline anhydrous aripiprazole crystals in an amount of about 2% to 10% by weight of the dissolved aripiprazole, preferably about 2% by weight. The addition of seeding is carried out at temperatures of about 70° C., preferably at about 70° C.-75° C.

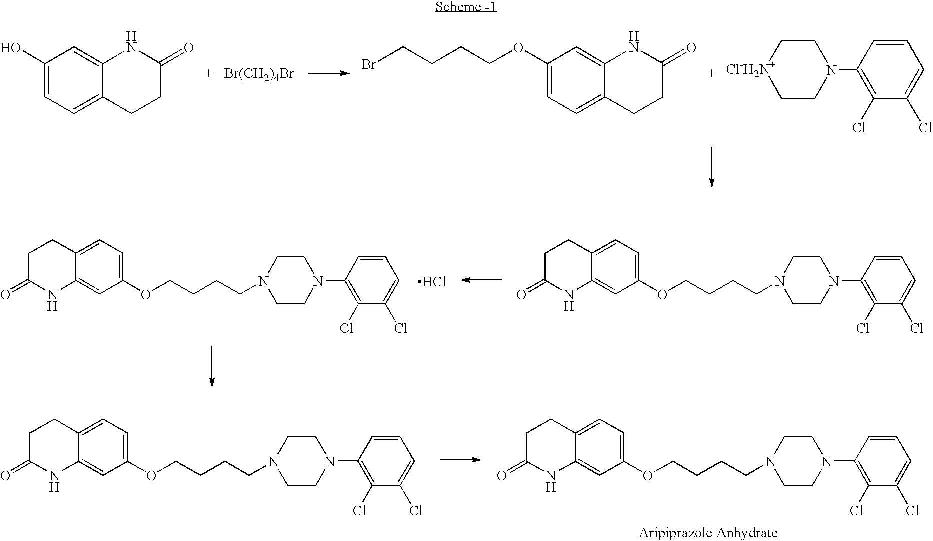

Crystalline anhydrous aripiprazole prepared according to the present invention has a mean particle size between 1-200 μm, preferably the mean particle size is between 150-200 μm, most preferably the mean particle size is between 160-170 μm, when measured by Malvern light scattering instrument.

Preferably, crystalline anhydrous Aripiprazole prepared according to the present invention has a mean particle size between 1-50 μm, preferably the mean particle size is between 1-10 μm, most preferably the mean particle size is between 1-5 μm after unit operation of jet-milling for one time as measured by Malvern light scattering instrument.

Preferably, crystalline anhydrous Aripiprazole prepared according to the present invention has a mean particle size between 1-50 μm, preferably the mean particle size is between 1-10 μm, most preferably the mean particle size is between 1-5 μm after unit operation of jet-milling for two times as measured by Malvern light scattering instrument.

Preferably, crystalline anhydrous Aripiprazole prepared according to the present invention is dried at about 100° C. till the water content is not more than 0.5% w/w.

An improved process of the present invention is a simple, cost effective, industrially scaleable and environment friendly process for the synthesis of crystalline anhydrous aripiprazole.

According to the second embodiment of the present invention, a process for preparing novel form of crystalline aripiprazole hydrochloride Form X comprising:
(i) providing a solution of aripiprazole in an organic solvent;
(ii) maintaining the solution of step (i) at ambient temperature;
(iii) optionally charcoalizing the solution of step (ii);
(iv) filtering the solution;
(v) treatment of the filtrate of step (iv) with an organic solvent;
(vi) treating the solution of step (v) with suitable hydrochloride agent;
(vii) isolating the product by filtration;
(viii) optionally washing the product with an organic solvent; and
(ix) drying the product to obtain Form X of crystalline aripiprazole hydrochloride.

Suitable organic solvents for preparing the solution can be selected from the group consisting of alcohols such as, methanol, ethanol, isopropanol, n-butanol and pentanol; or esters like ethyl acetate, methyl acetate, tert-butyl acetate, or ketones like acetone, methyl ethyl ketone, or chlorinated solvents like methylene dichloride, ethylene dichloride, chloroform etc, or tetrahydrofuran, or mixtures of any two or more thereof, preferably methylene dichloride.

According to an important aspect of the present invention, a solution of aripiprazole in methylene dichloride is not a clear solution which is maintained at an ambient temperature from about 20° C. to about 40° C., preferably about 25° C. to about 30° C.

According to yet another important aspect of the present invention, aripiprazole is isolated in a novel crystalline form of aripiprazole hydrochloride wherein hydrochloride agent is hydrochloride gas, alcoholic hydrochloride or aqueous hydrochloride, preferably alcoholic hydrochloride selected from methanolic hydrochloride, ethanolic hydrochloride or Isopropyl hydrochloride, most preferably isopropyl hydrochloride.

Aripiprazole hydrochloride prepared by the process disclosed herein above is dried at about 25° C. to about 35° C. for 2 hours followed by drying at about 65° C. to about 70° C. to obtain Form X of crystalline aripiprazole hydrochloride is also one of the embodiment of the present invention.

The present invention provides a novel crystalline aripiprazole hydrochloride “Form-X” characterized by x-ray powder diffraction pattern having 2θ values 3.84, 7.6, 8.59, 10.98, 11.62, 12.80, 13.37, 14.54, 15.35, 17.44, 18.14, 19.66, 22.02, 22.36, 23.11, 24.92, 25.58, 27.71, 26.68, 28.98, 29.44 & 30.16 as the characteristic peaks and having DSC endothermic peak at 223° C.

It will be appreciated that the illustrated process can be simply applied for the large-scale production of crystalline anhydrous aripiprazole.

Although the invention has been described with reference to a specific example, it will be appreciated by those skilled in the art that the invention may be embodied in many other forms.

The process of the present invention will be explained in more detail with reference to the following examples, which are provided by way of illustration only and should not be constructed as limit to the scope of the claims in any manner.

### EXAMPLES

### Example-1

### Preparation of 7-(4-bromobutoxy)-3,4-dihydrocarbostyryl

The solution of 7-(4-hydroxy)-3,4-dihydrocarbostyryl (125 g), potassium carbonate (158.75 g) and 1,4-dibromobutane (662.5 g) was prepared in acetone (1375 mL) and refluxed at 60° C.-65° C. for 16 hours. After the completion of the reaction, the reaction mixture was cooled to 15° C.-20° C. and stirred for 1.0 h. Isolated solid was filtered and washed with chilled acetone (250 mL). The filtrate was distilled under vacuum at 60° C. to remove access of acetone and at 120° C. to remove traces of 1,4-dibromobutane. The oily residue were cooled at 40° C.-45° C. and treated with methylene dichloride and stirred for 15-20 minutes till clear solution was obtained. The reaction mixture was treated with 5% NaOH and process water at 25° C.-35° C. and the organic layer was separated. The organic layer was further charcoalized and filtered through cilete bed. The filtrate was subjected to distillation under vacuum to remove excess of methylene dichloride. The resulting mass was treated with ethyl acetate (125 mL) and cyclohexane (500 mL) at 25° C.-35° C. and stirred for 1 h. The solid thus obtained was filtered, washed with cyclohexane and suck dried. The product was dried in hot air oven at 55° C.-60° C.

### Example-2

### Preparation of Crude Aripiprazole

The solution of 7-(4-bromobutoxy)-3,4-dihydrocarbostyryl (150 g), 1-(2,3-dichloro)piperazine hydrochloride (228.0 g) and potassium carbonate (150 g) was prepared in methanol (900 mL) and refluxed at 65° C.-70° C. for 6-8 hours. After the completion of the reaction, the reaction mixture was cooled to 25° C.-30° C. and stirred for 1.0 h. Isolated solid was filtered and washed with methanol (250 mL) followed by process water (250 mL). The wet cake and the process water (1500 mL) were heated at 55° C.-65° C. and stirred for 1 h. The solid thus obtained was filtered, washed with process water till the pH of washing mL was 6.5-7.5 and suck dried. The product was dried in hot air oven at 65° C.-70° C. till the moisture content was not more than 1.0% w/w.

### Example-3

### Preparation Aripiprazole Hydrochloride

The solution of aripiprazole crude (180.0 g) was prepared in methylene dichloride (1080 mL) and was stirred for 15 minutes at 25° C.-30° C. resulted in the hazy solution. The reaction mixture was charcoalized and stirred for 1.0 h. The charcoalized reaction mass was filtered and wash with methylene dichloride (360 mL). The filtrate was treated with methylene dichloride (720 mL) at 25° C.-30° C. and stirred for 15 minutes. The solution of IPA:HCl (20% w/v) (219.6 g) was added slowly at 25° C.-35° C. and reaction mass was stirred for 1 h. The reaction mass was filtered and washed with methylene dichloride (360 mL). The product was air dried for 2 hrs at 25° C.-35° C. followed by drying in hot air oven at 65° C.-70° C.

### Example-4

### Preparation Aripiprazole Crude

The solution of aripiprazole hydrochloride (170.0 g) was prepared in methanol (3060 mL) and was heated to reflux at 65° C.-70° C. till clear solution is obtained. The reaction mixture was stirred for 15 minutes and cooled to 50° C.-55° C. The reaction mixture was charcoalized and stirred for 1.0 h, filtered and wash with methanol (340 mL) at 45° C.-55° C. The filtrate was treated with sodium hydroxide solution (112.2 g in 510 mL of water) at 40° C.-45° C., cooled to 20° C.-25° C. and stirred for 1 h. The reaction mass was filtered and washed with process water till the pH of the washing mL was 6.0-8.0 and suck dried. The product was dried in hot air oven at 65° C.-70° C. till water content not more than 1.0% w/w.

### Example-5

### Preparation Crystalline Anhydrous Aripiprazole

Aripiprazole Crude (130 g) prepared as per example 4 was refluxed in ethanol (1300 mL) at 78° C.-85° C. till the clear solution was obtained and stirred for 15-20 minutes. The reaction mixture was gradually cooled to 25° C.-35° C. and further to 5° C.-10° C. The isolated product was filtered and washed with chilled ethanol (130 mL). The wet solid was further treated with ethanol (1950 mL) and refluxed for 78° C.-85° C. The reaction mixture was charcoalized, filtered and washed with hot ethanol (260 mL). The filtrate was heated to reflux at 78° C.-85° C. till clear solution was obtained. The solution was stirred for 15-20 minutes and cooled to 70° C. The seeding material of crystalline anhydrous aripiprazole (2.6 g) was added to the reaction mixture. The reaction mixture was gradually cooled to 25° C.-35° C. and further to 5° C.-10° C. The isolated product was filtered and washed with ethanol (130 mL). The wet product was suck dried for 15-30 minutes. The product thus obtained was further dried at 100° C. for 18 hours till LOD and water content not more than 0.5% in hot air oven to give stable aripiprazole anhydrous crystalline.

1. A process for preparing crystalline anhydrous aripiprazole comprising the steps of:
(i) dissolving aripiprazole in an organic solvent to obtain a solution;
(ii) heating the solution from step (i) at about 70° C. to about 90° C. to obtain a clear solution;
(iii) cooling the solution of step (ii) to about 25° C. to about 35° C., further to about 5° C. to about 10° C. and maintaining the temperatures as crystals form;
(iv) separating crystals from the solution;
(v) dissolving the wet crystals in an organic solvent;
(vi) heating the solution from step (v) at about 70° C. to about 90° C. to obtain clear solution;
(vii) optionally, charging 5%-10% decolorizing carbon to the clear solution of step (iv) and stirring at the heating temperature for 15-60 minutes;
(viii) removing of the carbon under the hot condition;
(ix) heating the filtrate of step (viii) under stirring to about 70° C. to about 90° C. to obtain a clear solution;
(x) seeding the solution of step (ix) with crystalline anhydrous Aripiprazole at temperatures of about 70° C.;
(xi) cooling the solution of step (x) to about 25° C. to about 35° C.; further to about 5° C. to about 10° C. and maintaining the temperatures as crystals form;
(xii) filtering the separated solid in step (xi) further to about 0° C. to about 10° C. and washing the solid with organic solvent; and
(xiii) drying the solid of step (xii) at about 100° C. until the water content is not more than 0.5% w/w.

2. The process as claimed in claim 1, wherein in step (i) the organic solvent is selected from the group consisting of alcohols such as methanol, ethanol, isopropanol, n-butanol and pentanol; or esters like ethyl acetate, methyl acetate, tert-butyl acetate, or acetic acid, or tetrahydrofuran, or mixtures of any two or more thereof.

3. The process as claimed in claim 2, wherein the organic solvent is ethanol.

4. The process as claimed in claim 1, wherein in step (ii) said aripiprazole dissolved in an organic solvent is heated at about 78° C. to about 85° C. to obtain the clear solution.

5. The process as claimed in claim 1, wherein seeding the solution of step (ix) comprises adding crystalline anhydrous aripiprazole crystals in an amount of about 2% to 10% by weight of the dissolved aripiprazole

6. The process as claimed in claim 5, wherein crystalline anhydrous aripiprazole is added to about 2% by weight of the dissolved aripiprazole.

7. The process as claimed in claim 1, wherein the addition of seeds is carried out at temperatures of about 70° C., preferably at about 70° C.-75° C.

8. A crystalline anhydrous aripiprazole prepared as claimed in claim 1, characterized by DSC endothermic peak at 139° C.

9. The crystalline anhydrous aripiprazole as claimed in claim 8, characterized by DSC as substantially as disclosed in FIG. I.

10. A crystalline anhydrous aripiprazole prepared as claimed in claim 1, characterized by X-ray powder diffraction pattern having 2θ values 10.9, 14.2, 16.4, 19.2, 20.2, 21.9 measured on Rigaku D/Max-2200/PC Diffractometer with Cu K alpha-1 radiation source.

11. The crystalline anhydrous aripiprazole as claimed in claim 10, characterized by X-ray powder diffraction pattern having 2θ values 8.6, 10.9, 11.9, 13.6, 14.2, 14.8, 16.4, 17.5, 19.2, 20.2, 21.9, 24.6, 26.4, 28.1 measured on Rigaku D/Max-2200/PC Diffractometer with Cu K alpha-1 radiation source.

12. The A crystalline anhydrous aripiprazole as claimed in claim 10, characterized by X-ray powder diffraction pattern as substantially as disclosed in FIG. II.

13. A crystalline anhydrous aripiprazole prepared as claimed in claim 1, having a mean particle size between 1-200 μm, preferably between 150-200 μm, most between 160-170 μm, when measured by Malvern light scattering instrument.

14. A crystalline anhydrous aripiprazole prepared as claimed in claim 1, having a mean particle size between 1-50 μm, preferably between 1-10 μm, most preferably between 1-5 μm after unit operation of jet-milling for one time as measured by Malvern light scattering instrument.

15. A crystalline anhydrous aripiprazole prepared as claimed in claim 1, having a mean particle size between 1-50 μm, preferably between 1-10 μm, most preferably between 1-5 μm after unit operation of jet-milling for two time as measured by Malvern light scattering instrument.

16. A process for preparing novel form of crystalline aripiprazole hydrochloride Form X comprising:
(i) providing a solution of aripiprazole in an organic solvent;
(ii) maintaining the solution of step (i) at ambient temperature;
(iii) optionally charcoalizing the solution of step (ii);
(iv) filtering the solution;
(v) treatment of the filtrate of step (iv) with an organic solvent;
(vi) treating the solution of step (v) with a suitable hydrochloride agent;
(vii) isolating the product by filtration;
(viii) optionally washing the product with an organic solvent; and
(ix) drying the product to obtain Form X of crystalline aripiprazole hydrochloride.

17. The process as claimed in claim 16, wherein in step (i) said an organic solvent is selected from the group consisting of alcohols such as methanol, ethanol, isopropanol, n-butanol and pentanol; or esters like ethyl acetate, methyl acetate, tert-butyl acetate, or ketones like acetone, methyl ethyl ketone, or chlorinated solvents like methylene dichloride, ethylene dichloride, chloroform etc, or tetrahydrofuran, or mixtures of any two or more thereof.

18. The A process as claimed in claim 16, wherein in step (i) the solution of aripiprazole in an organic solvent is not a clear solution.

19. The A process as claimed in claim 18, wherein said organic solvent is methylene dichloride.

20. The A process as claimed in claim 16, wherein in step (ii) said ambient temperature is from about 20° C. to about 40° C., preferably about 25° C. to about 30° C.

21. The A process as claimed in claim 16, wherein in steps (v) and (viii) said organic solvent is methylene chloride.

22. The A process as claimed in claim 16, wherein in step (vi) said hydrochloride agent is hydrochloride gas, alcoholic hydrochloride or aqueous hydrochloride.

23. The A process as claimed in claim 22, wherein said hydrochloride agent is alcoholic hydrochloride selected from methanolic hydrochloride, ethanolic hydrochloride or Isopropyl hydrochloride preferably isopropyl hydrochloride.

24. The A process as claimed in claim 16, wherein in step (ix), said drying is done at about 25° C. to about 35° C. for 2 hours followed by drying at about 65° C. to about 70° C. to obtain Form X of crystalline aripiprazole hydrochloride.

25. A novel form of crystalline aripiprazole hydrochloride Form-X characterized by x-ray powder diffraction pattern having 2θ values 7.6, 10.9, 12.8, 15.3, 18.4, 22.0, 27.7 measured on Rigaku D/Max-2200/PC Diffractometer with Cu K alpha-1 radiation source.

26. A novel form of crystalline aripiprazole hydrochloride Form-X as claimed in claim 25, further characterized by x-ray powder diffraction pattern having 2θ values 3.8, 7.6, 8.5, 10.9, 11.6, 12.8, 13.3, 14.5, 15.3, 17.4, 18.1, 19.6, 22.0, 22.3, 23.1, 24.9, 25.5, 27.7, 26.6, 28.9, 29.4 and 30.1 measured on Rigaku D/Max-2200/PC Diffractometer with Cu K alpha-1 radiation source.

27. A novel form of crystalline aripiprazole hydrochloride as claimed in claim 25, characterized by X-ray powder diffraction pattern as substantially as disclosed in FIG. III.

28. A novel form of crystalline aripiprazole hydrochloride Form-X as claimed in claim 25, further characterized by differential scanning calorimetry endothermic peak at 223° C.

29. A novel form of crystalline aripiprazole hydrochloride as claimed in claim 28, characterized by DSC as substantially as disclosed in FIG. IV.
